(19) United States
(12) Patent Application Publication
(10) Pub. No.: US 2025/0120779 A1
(43) Pub. Date: Apr. 17, 2025

(54) METHOD FOR CONTROLLING AN ARTICULATED END EFFECTOR ACTUATED BY ONE OR MORE ACTUATION TENDONS OF A SURGICAL INSTRUMENT OF A ROBOTIC SYSTEM FOR SURGERY, AND RELATED ROBOTIC SYSTEM FOR SURGERY

(71) Applicant: MEDICAL MICROINSTRUMENTS, INC., Wilmington, DE (US)

(72) Inventors: Matteo TANZINI, Pisa (IT); Matteo BAGHERI GHAVIFEKR, Pisa (IT); Antonio DI GUARDO, Pisa (IT); Giuseppe Maria PRISCO, Pisa (IT); Massimiliano SIMI, Pisa (IT)

(21) Appl. No.: 18/694,702

(22) PCT Filed: Sep. 21, 2022
(86) PCT No.: PCT/IB2022/058920

§ 371 Date: Mar. 22, 2024

(30) Foreign Application Priority Data
Sep. 23, 2021 (IT) 102021000024434

(51) Int. Cl. A61B 34/00; A61B 34/35; A61B 90/00; G16H 20/40; G16H 40/63

(52) U.S. Cl. CPC A61B 34/71; A61B 34/35; G16H 20/40; G16H 40/63; A61B 2090/064

(57) ABSTRACT

A method controls an articulated end effector actuated by actuation tendon(s) of a surgical instrument of a robotic surgery system. The surgical instrument has an articulated end effector and an actuation tendon to actuate the end effector. A controller and a motorized actuator are operatively connectable to a respective actuation tendon to impart an action, controlled by the controller, to the respective actuation tendon, to determine a univocal correlation between movement of motorized actuator(s) and a respective movement of the articulated end effector. A force exerted by the motorized actuator(s) during the operating step is detected. A length variation of the actuation tendon(s) due to elastic elongation of the actuation tendon is estimated based on a detected force; and then the estimated length variation is used for position control, which moves the motorized actuator(s) taking into account the estimated length variation to reduce or cancel error from elastic elongation.

### CROSS-REFERENCE TO RELATED APPLICATIONS

This application is a National Phase of International Application No. PCT/IB2022/058920, filed on Sep. 21, 2022, which claims priority to Italian Patent Application No. 102021000024434, filed on Sep. 23, 2021, the entire contents of which are incorporated into this application by reference.

### FIELD OF APPLICATION

The present invention relates to a method for controlling an articulated end effector actuated by means of one or more actuation tendons of a surgical instrument of a robotic system for surgery, and the related robotic system for surgery.

In particular, the invention relates to a control method which provides a compensation for the position error of a joint with respect to the commanded position.

Therefore, the present description more generally relates to the technical field of operational control of robotic systems for teleoperated surgery.

### PRIOR ART

Known robotic systems for medicine and/or surgery typically comprise at least one articulated terminal (or “articulated end effector” or “end effector”) intended to interact with an anatomy of a patient, whether to perform surgical or microsurgical procedures such as sutures, anastomoses, incisions, or to acquire images, or diagnostic information.

The articulated end effector is typically actuated by actuation cables (tendons) which transfer a traction action to the articulated end effector.

Robotic systems for medicine and/or surgery can operate according to a master-slave control architecture, for example where the master is hand-held by a surgeon, or they can operate in autonomous mode, for example by performing a series of programmed operations.

Anthropomorphic robotic systems are also known where the articulated end effector comprises anthropomorphic joints, such as the joints of the phalanges of a robotic hand, which are implemented by traction action applied on actuation tendons.

The robotic system motors can be placed upstream of the articulated end effectors, and the actuation tendons are operatively connected to both the motors and the articulated end effector. The pose of the articulated end effector is determined by the action of the motors of the robotic system which is transmitted by the actuation tendons.

The number of actuation tendons for the movement of a plurality of degrees of freedom can vary, but typically two antagonistic tendons are connected to the same degree of freedom of the articulated end effector to move it in opposite directions.

Therefore, the need to provide a solution capable of ensuring the correspondence between the action of the motors and the pose assumed by the articulated end effector is strongly felt.

In fact, the tendons can be in mutual sliding contact when in operating conditions, as well as they can roll up, i.e., intertwine with each other, or they can slide on the walls of the articulated end effector or on the walls of a rigid or flexible or articulated positioning shaft. These conditions can affect the accuracy of the transfer of the action of the motors to the articulated end effector, resulting in a mismatch between the action of the motors and the pose of the articulated end effector.

In other words, the expected pose may not be achieved due to the distortion of the action imparted by the motors due to the mechanical behavior of the actuation tendons.

In addition, in the case of miniaturized articulated end effectors, the sizing of the actuation tendons becomes decisive for transferring the action of the motors to the articulated end effector. In fact, as the scale decreases, a longitudinal deformation of the tendon which is recoverable by a precise amount becomes increasingly significant.

To facilitate the miniaturization of the articulated end effector, it is possible to resort to the use of polymeric actuation tendons, as shown for example by WO-2017-064303 and US-2021-0106393 on behalf of the same Applicant.

Such types of actuation tendons allow to reduce tendon friction and diameter, allowing to travel very small connecting radii.

Furthermore, miniaturized articulated end effectors are typically arranged at the distal end of a positioning shaft which forces the actuation tendons to extend for relatively long stretches in relation to the extent of the tendon stretch along the articulated end effector device only at the distal end of the shaft. The provision of such long, thin tendons increases the occurrence of deformability of the tendon in the longitudinal direction, when in operating conditions.

For example, in winch transmission systems, the tendons wind on a rotating spool and can intersect with each other, i.e., intertwine during such winding, locally increasing friction and potentially causing a tearing transmission of the action of the motors.

Similarly, where the tendons are intertwined i.e., interwoven inside the extension of the shaft of a medical and/or surgical instrument, an increase in friction which would affect the transmission would occur locally.

In other words, in the cases mentioned above, there would be a mismatch between the action of the motors and the movement of the articulated end effector due to sliding friction phenomena between different tendons or between sections of the same tendon.

Another situation which could generate a mismatch between the action of the motors and the movement of the articulated end effector could arise from the intrinsic elasticity of the individual actuation tendons, which could lengthen when stressed, absorbing a part of the action imparted by the respective motors without effectively transferring it to the articulated end effector. Typically, the elastic recovery of the deformation occurs quickly when the perturbation ceases, and in the specific case, it is possible for the tendons to immediately recover the elastic deformation thereof when the action imparted by the motors ceases.

However, these dynamics could transfer an unwanted movement to the articulated end effector. For example, a rotary joint of an articulated wrist could be activated when the motors stop acting.

Miniaturized articulated end effectors are desirable in the medical-surgical sector, as well as in the field of anthropomorphic robots, as well as in micro-electronics, micro-mechanics, precision mechanics, watchmaking, jewelry and costume jewelry and more generally in automation.

Particularly in the medical-surgical field, the articulated end effector is a sterile component of the system and works in the sterile field when under operating conditions and it is often not possible or not desirable to equip the articulated end effector with an active sensor system to allow the robotic system to detect the pose assumed by the articulated end effector itself in real time.

At the same time, the push towards extreme miniaturization of the articulated end effectors is strongly felt in this field and a smart control on the position of the articulated end effector and on the action performed by the articulated end effector is necessary to ensure safety and at the same time usability.

In the case of a robotic system remotely operated according to a master-slave control architecture, the action of the motors is controlled based on the action imparted by the user on a master control device. The master control device can be in the form of a joystick, i.e., a mechanical attachment projecting cantilevered from a master operating console, and can comprise a motorized force feedback system that returns tactile feedback to the user which depends on the information detected by the sensor system of the articulated end effector.

Teleoperated robotic systems are also known in which the master control device is “ungrounded”, i.e., not constrained to the ground in which it is possible to not include a tactile feedback system.

Therefore, the need to devise a solution to ensure the correspondence between the action imparted by the motors of a robotic system and the action performed by the articulated end effector, avoiding including sensor systems on the articulated end effector itself, is very felt in different areas.

At the same time, there is a need to miniaturize articulated end effectors of robotic systems without resulting in decreased control over the articulated end effector itself.

### SUMMARY OF THE INVENTION

It is the object of the present invention to provide a method for controlling an articulated end effector, actuated by actuation tendons of a surgical instrument of a robotic system for surgery, which allows at least partially overcoming the drawbacks complained above with reference to the background art, and to respond to the aforementioned needs particularly felt in the technical field considered.

It is further the object of the present invention to provide a robotic system for surgery adapted to be controlled by the aforesaid control method, and/or configured to carry out the aforesaid method.

The aforesaid method provides solutions to the technical problems mentioned above.

In fact, by virtue of the proposed solutions, it is possible to use a behavior model in operating conditions of an actuation tendon to create a position control method of a motor of a robotic manipulator aimed at actuating a miniaturized articulated end effector.

The motor position control is a feedback-operated control loop based on the information detected on the force imparted by the motor to a transmission unit comprising at least said actuation tendon.

The information on the imparted force can be detected by a load cell placed on the motor at the interface with the transmission unit. For example, the transmission unit comprises a rigid element, for example a piston, which interfaces with the motor and an actuation tendon connected to the articulated end effector and rigidly connectable to the rigid element, for example glued to the piston. Thereby, the force detected at the interface between the motor and the rigid element of the transmission unit rigidly connectable to the actuation tendon is substantially equal to the traction force applied on the actuation tendon. Where the connection between the motor and the actuation tendon is not rigid, the method could consider the yielding of the connection between motor and tendon.

The information on the force imparted is used for the real-time estimation of the elastic elongation of the actuation tendon. For example, the elastic elongation of the actuation tendon can be proportional to the force imparted by the motor to the transmission unit, as detected.

By virtue of the proposed solutions, it is possible to ensure the correspondence between the action of the motors and the pose assumed by the articulated end effector, avoiding adding sensors to the articulated end effector, keeping “upstream”, as much as possible, the sensors for detecting useful information for the control method. For example, in the medical-surgical field this allows keeping the sensors out of the sterile field.

Where a teleoperated master-slave robotic system is provided, the proposed solutions help ensuring the correspondence between the action of the master device and the pose assumed by the articulated end effector of the slave device, minimizing master-slave tracking delay.

By virtue of the proposed solutions, it is possible to compensate for an elastic component of said mismatch between the action of the motor and the pose assumed by the articulated end effector.

By virtue of the proposed solutions, it is possible to use, in a precise and controlled manner, actuation tendons that are long, thin and subject to a high elastic deformation, based on the force imparted by the motors.

By virtue of the proposed solutions, it is possible to accurately estimate the current pose of the articulated end effector by means of a model of the transmission action performed by the transmission unit based on the force imparted to the transmission unit, as detected.

The method according to the invention is particularly adapted but not uniquely intended to control a robotic system for surgery, not necessarily of the master-slave type.

The method according to the invention is adapted to control an anthropomorphic robotic system not necessarily comprising robotic phalanges actuated by actuation tendons.

### BRIEF DESCRIPTION OF THE DRAWINGS

Further features and advantages of the method according to the invention will become apparent from the following description of preferred exemplary embodiments, given by way of non-limiting indication, with reference to the accompanying drawings, in which:

FIG. 1 shows in axonometric view a robotic system for teleoperated surgery, according to an embodiment;

FIG. 2 shows in axonometric view a portion of the robotic system for teleoperated surgery of FIG. 1;

FIG. 3 shows in axonometric view a distal portion of a robotic manipulator, according to an embodiment;

FIG. 4 shows in axonometric view a surgical instrument, according to an embodiment, in which tendons are diagrammatically shown in a dashed line;

FIG. 5 diagrammatically shows the actuation of a degree of freedom of an articulated end effector of a surgical instrument, according to a possible operating mode;

FIGS. 6, 7, 7bis and 7ter show operating aspects of an embodiment of the control method according to the invention;

FIG. 8 is a flow diagram showing steps of a conditioning method, according to a possible operating mode;

FIG. 9 diagrammatically shows a motorized actuator, a transmission element and a tendon of a surgical instrument, according to an embodiment;

FIG. 10 is a diagrammatic sectional view of a portion of a surgical instrument and a portion of a robotic manipulator showing the actuation of a degree of freedom of a surgical instrument, according to a possible operating mode;

FIG. 11 is a partially sectioned axonometric view for clarity showing an articulated end effector of a surgical instrument, according to an embodiment;

FIG. 12 depicts a block diagram, in the time domain, of a control/compensation method according to an embodiment of the present invention;

FIGS. 12bis and 12ter depict two block diagrams, in the Z transform domain, in two different conditions, of a control/compensation method according to an embodiment of the present invention;

FIGS. 13 and 14 show some operating conditions and/or states of the surgical instrument on which the execution of the control/compensation method according to the invention can be applied or inhibited, in accordance with various possible operating modes.

### DETAILED DESCRIPTION

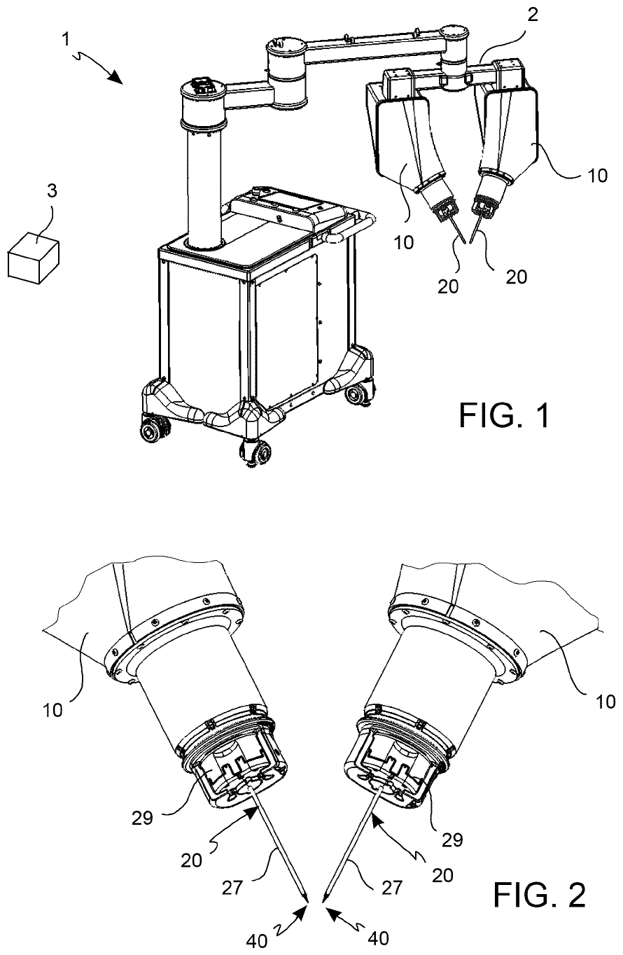

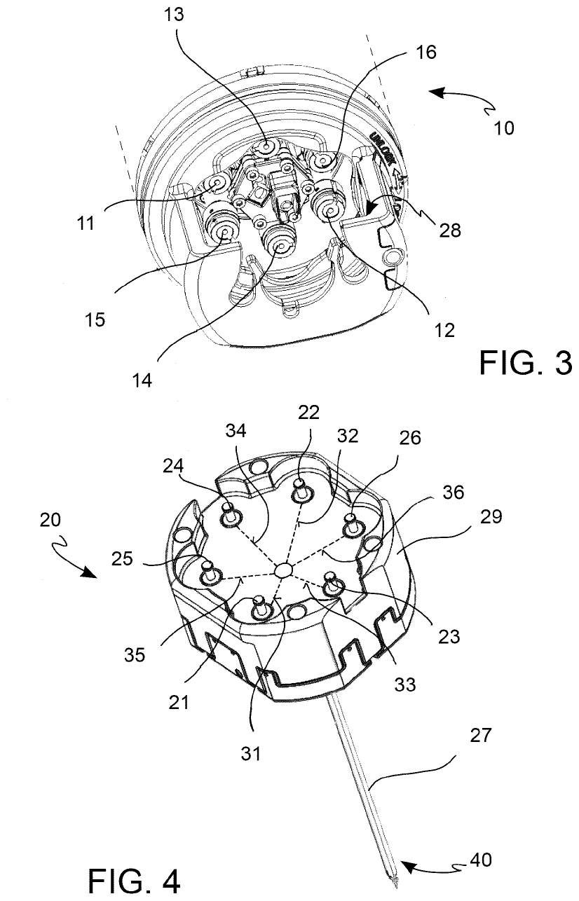

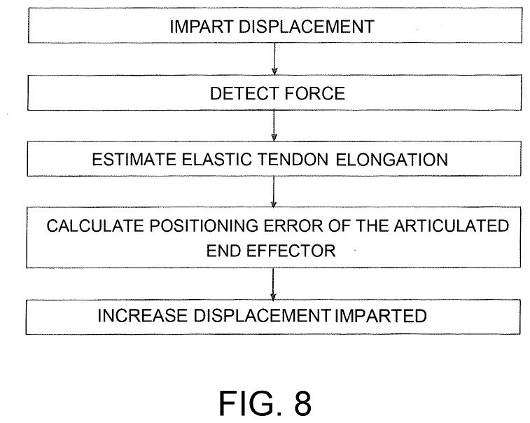

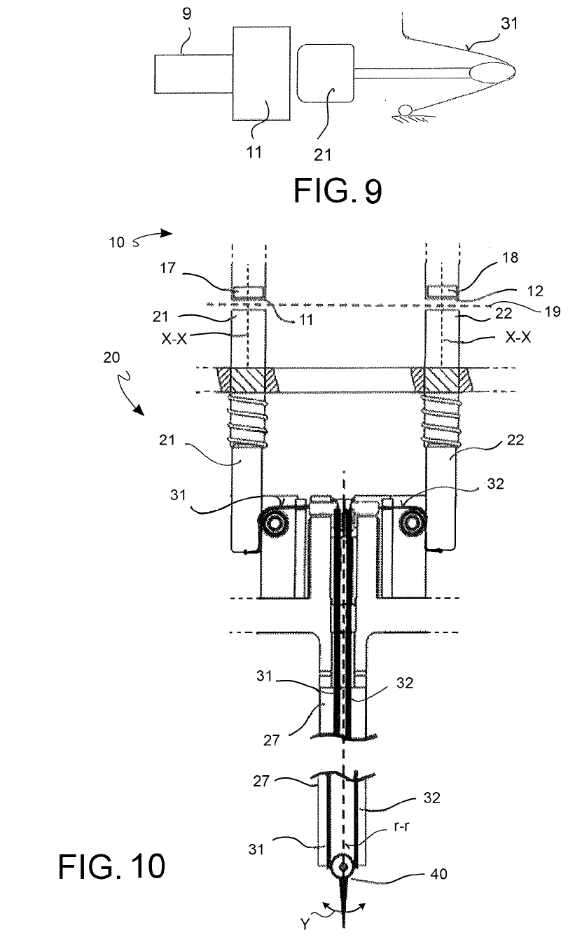

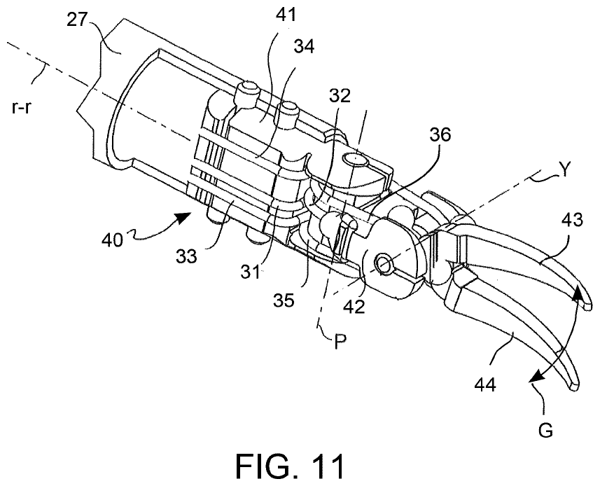

With reference to FIGS. 1-14, a method is described for controlling an articulated end effector 40 actuated by one or more actuation tendons of a surgical instrument 20 of a robotic system for surgery.

The articulated end effector will hereinafter also be referred to as “articulated end device” or “end effector” (commonly used English terminology).

The method is advantageously executable during an operating phase of the surgical instrument.

The method is applied to a surgical instrument 20 comprising an articulated end effector 40 and at least one actuation tendon 31, 32, 33, 34, 35, 36, configured to actuate the articulated end effector 40.

The method is applied to a robotic system for surgery comprising, in addition to said surgical instrument 20, control means 9 and at least one motorized actuator 11, 12, 13, 14, 15, 16, operatively connectable to a respective said at least one actuation tendon 31, 32, 33, 34, 35, 36 to impart an action to the respective actuation tendon, controlled by the control means 9, so as to determine a univocal correlation between at least one movement of one or more motorized actuators 11, 12, 13, 14, 15, 16 and a respective at least one movement of the articulated end effector 40.

The method first comprises the step of detecting the force Fm exerted by at least one of the aforesaid one or more motorized actuators 11, 12, 13, 14, 15, 16, during the aforesaid operating phase of the surgical instrument.

The method then comprises the steps of estimating, by means of a predefined mathematical model, based on the detected force Fm, a length variation of at least one of the one or more actuation tendons 31, 32, 33, 34, 35, 36, due to elastic elongation of the actuation tendon; and then using the estimated length variation for a position control of the one or more motorized actuators 11, 12, 13, 14, 15, 16.

Such a position control comprises imparting a movement on the aforesaid at least one motorized actuator 11, 12, 13, 14, 15, 16, taking into account the estimated length variation of said at least one actuation tendon 31, 32, 33, 34, 35, 36, so as to reduce or cancel the error introduced by said elastic elongation between the position reached by the articulated end effector 40 and a desired nominal position of the articulated end effector 40.

Such a desired nominal position, for example, can be that position which would be obtained in the absence of elastic elongation.

It should be noted that the aforesaid technical effect of reducing or cancelling the error introduced by the elastic elongation can comprise or correspond to a “compensation” for such error and/or a “minimization” of such error.

In accordance with an embodiment, in which the robotic system is a master-slave system in which the surgical instrument is a slave device commanded, according to a control mode, by a master device of the robotic system, the method allows achieving a predetermined kinematic congruence between the pose commanded by the master device and the pose reached by the articulated end effector 40 of the slave device (i.e., in the absence of external forces, it allows minimizing, in a finite time, the error between the pose commanded by the master device and the pose reached by the articulated end effector 40 of the slave device).

In accordance with an embodiment of the method, in which the robotic system is a master-slave system in which the surgical instrument is a slave device controlled, according to a control mode, by a master device of the robotic system, the step of imparting takes into account the command action performed by the user.

According to alternative implementations, the method applies to an autonomous robotic system without a master device or with a master device temporarily or permanently deactivated.

According to an implementation option, the method applies to an unconstrained master device (i.e., “flying” or “groundless”).

According to an implementation option, the method is applied to a master device without a force feedback system, whereby the user does not receive information from the master device.

In accordance with an embodiment of the method, in which the surgical instrument 20 comprises a plurality of actuation tendons 31, 32, 33, 34, 35, 36, and the robotic system for surgery comprises a respective plurality of motorized actuators 11, 12, 13, 14, 15, 16, the aforesaid step of detecting a force is carried out on a plurality or on all the motorized actuators 11, 12, 13, 14, 15, 16; the aforesaid step of estimating is performed with reference to a plurality or all actuation tendons 31, 32, 33, 34, 35, 36; the aforesaid step of imparting is performed on a plurality or on all motorized actuators 11, 12, 13, 14, 15, 16.

In accordance with an embodiment, the method comprises the further step of verifying information related to the state of the robotic system; then deciding, by the control means 9, whether or not to perform the aforesaid step of imparting a movement on a motorized actuator, to reduce and/or cancel and/or compensate for the error introduced by the elastic elongation, based on one or more conditions related to the state of the robotic system; and performing the imparting step only if the aforesaid one or more conditions are met.

According to an embodiment, the method is applied to a robotic system with a hand-held, unconstrained, master device adapted to be moved by an operator and to be manipulated by the operator according to a degree of freedom associated with the closing and/or gripping of the microsurgical slave instrument.

In an implementation option of such an embodiment, it is provided that when, during a teleoperation, the surgical instrument is in a gripping state, the aforesaid step of imparting a movement on a motorized actuator, to reduce and/or cancel and/or compensate for the error introduced by the elastic elongation, is inhibited for at least one of the motorized actuators connected to a respective at least one actuation tendon of a gripping degree of freedom.

In another implementation option of such an embodiment, it is provided that when, during a teleoperation, the surgical instrument is in a gripping state, the aforesaid step of imparting a movement on a motorized actuator is decreased according to a scaling factor between 0 and 1, for at least one of the motorized actuators connected to a respective at least one actuation tendon of a gripping degree of freedom.

In another implementation option of such an embodiment, it is provided that the aforesaid step of imparting a movement on a motorized actuator is inhibited for the two motorized actuators connected to the respective two antagonistic actuation tendons of the grip closing degree of freedom, or for the four motorized actuators connected to the four actuation tendons of the pairs of antagonistic actuation tendons of the grip closing and grip opening degrees of freedom.

In another implementation option of such an embodiment, it is provided that the aforesaid step of imparting a movement on a motorized actuator is decreased according to a scaling factor between 0 and 1, for the two motorized actuators connected to the respective two antagonistic actuation tendons of the grip closing degree of freedom, or for the four motorized actuators connected to the four actuation tendons of the pairs of antagonistic actuation tendons of the grip closing and grip opening degrees of freedom.

In another implementation option of such an embodiment, it is provided that the aforesaid step of imparting a movement on a motorized actuator is inhibited for all the motorized actuators.

In another implementation option of such an embodiment, it is provided that the aforesaid step of imparting a movement on a motorized actuator is decreased according to a scaling factor between 0 and 1, for all the motorized actuators.

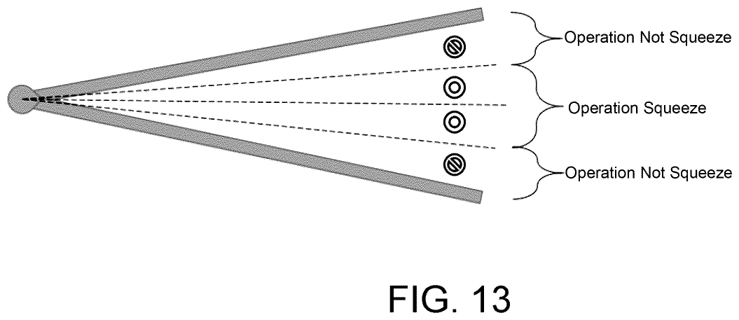

According to an implementation option, the method is performed in an operating phase (defined here as “not squeeze”, shown in FIG. 13), in which the teleoperation is active and the operator moves the degree of freedom of the master device associated with the closing and/or gripping of the microsurgical slave instrument, within the interval of master movement transferred in corresponding movement of the end effector and not of gripping force (closing angle greater than a certain threshold),

In contrast, the compensation method is inhibited in a phase (defined here as “freeze”) in which the teleoperation is active and the operator keeps the master beyond the gripping threshold (with a closing angle less than a certain threshold—“squeeze” condition shown in FIG. 13), while maintaining the compensation value at the same level as when the “freeze” step was entered.

According to an embodiment, in which the master device is a hand-held, unconstrained master device adapted to be moved by an operator and to be manipulated by the operator according to a degree of freedom associated with the closing and/or gripping of the microsurgical slave instrument, it is provided that, at the end of a teleoperation, when the surgical instrument is in a gripping state and it is desired to maintain the gripping state (condition defined here as “hold squeeze”), the aforesaid step of imparting a movement on a motorized actuator, is inhibited for all the motorized actuators connected to respective actuation tendons.

In accordance with an embodiment, the method provides that, when exiting the teleoperation in a non-gripping state, before re-entering a new teleoperation, the estimated length variation for each of the one or more actuation tendons, during the previous teleoperation, is removed.

According to an implementation option, in such conditions the elastic compensation actions are removed, thereby returning the surgical instrument to a known initial zero position.

In accordance with an embodiment, each of the aforesaid one or more actuation tendons 31, 32, 33, 34, 35, 36 is operatively connected both to a respective motorized actuator of the robotic surgical system and to the aforesaid articulated end effector 40, to actuate a respective degree of freedom among the one or more degrees of freedom (P, Y, G) of the articulated end effector 40.

According to an implementation option, the degrees of freedom of the articulated end effector 40 comprise a pitch degree of freedom, and/or a yaw degree of freedom, and/or a grip degree of freedom.

In accordance with an implementation option, at least one of said one or more actuation tendons 31, 32, 33, 34, 35, 36 actuates a rotational degree of freedom of the articulated end effector 40.

In accordance with an embodiment, the aforesaid step of detecting the force exerted by a motorized actuator 11, 12, 13, 14, 15, 16 is performed by a respective force sensor or torque sensor operatively connected to the respective motorized actuators.

According to an implementation option, such sensors are force sensors located on the contact interface of the respective motors (e.g., on the sterile side).

According to an implementation option, such sensors are torque sensors.

In accordance with an embodiment, the step of detecting a force Fm is performed continuously, with a detection frequency Fr, and the aforesaid position control of the one or more motorized actuators is performed continuously, with a position control frequency Fcp.

The aforesaid detection frequency Fr and position control frequency Fcp are set so as to ensure a compensation of the elastic elongation substantially in real time, with respect to the actuation times of the teleoperation, i.e., in real time, with a dynamics which cannot be perceived by the user.

According to an implementation option, the aforesaid detection frequency Fr and position control frequency Fcp coincide, and are comprised in an interval between 100 Hz and 1000 Hz.

In such a case, therefore, the compensation method is carried out at each period T, comprised in the interval between 1 and 10 ms, based on a force Fm detected at the same period.

In accordance with an embodiment, the step of estimating comprises estimating the length variation of an actuation tendon as the ratio between the modulus of the force detected Fm on the actuation tendon and an effective elastic constant value K which can be determined experimentally, or calculated or predetermined so as to ensure system response stability.

In accordance with an embodiment, the step of using the estimated length variation for a position control and the step of imparting a movement on the respective motorized actuator are carried out based on the formula:

$$u = \frac{Fm}{Ω Kel}$$

in which such a formula is specific for each motorized actuator and therefore such as to determine a specific control on each motorized actuator.

In the formula above, u is the position that is commanded to the motorized actuator, Kel is the elastic constant of the actuation tendon (hereinafter, such an elastic constant, if experimentally determined, will also be indicated as K_exp), Ω is a multiplicative parameter.

According to an implementation option, the multiplicative parameter Ω is greater than 1, so that the effective elastic constant value K=Ω Kel used in the calculation is greater than the tendon elastic constant value by a factor equal to the aforesaid multiplicative parameter Ω, thus ensuring that the effective tendon elastic constant value K is overestimated, and thus greater, than the elastic constant Kel of the same actuation tendon.

In the aforesaid embodiment, an important aspect relates to the ratio between the experimentally identified elastic constant (Kel, or K_exp) and the elastic constant K used within the model, also defined as “effective elastic constant value K”.

In particular, in such an embodiment, as shown above, the value K used in the algorithm must be greater than the experimentally determined value, k_exp, for convergence needs of the algorithm.

The aforesaid parameter SI defines a ratio between K and K_exp comprised in an interval between 100% and 150%, and preferably from +10% to +50%.

According to an implementation option, such a multiplicative parameter is between 0.7 and 1.5.

According to an implementation option of the aforesaid embodiments, the aforesaid effective elastic constant value K, and therefore also the multiplicative parameter Ω, are determined in a variable manner, depending on the state of the robotic system, and/or on the spatial conditions of the master device and/or the slave device and/or on the teleoperation permanence time.

In other words, K can vary depending on the teleoperation permanence time, or depending on the point of the (master or slave) workspace in which it is located; or to vary a compromise between system congruence and stability. The value of K can be re-estimated during teleoperation: for example, with large sudden increases in force, the value of K can be varied/adjusted for stability needs.

The value of K can be adjusted empirically, regardless of the elastic rigidity value of the actual tendon. For example, the value of K can be chosen on an experimental basis to account for the dispersions of the tendon-instrument system (e.g., due to local sliding frictions on a stretch of the tendon) and thus the value of K is not necessarily related to the actual elastic constant of the tendon when considered alone.

According to an implementation option, the value of K is an underestimate of the tendon elastic constant value if considered alone.

According to an implementation option, the value of K is chosen experimentally in an arbitrary manner in order to experimentally ensure system stability.

According to an embodiment, the compensation method is performed in the presence of detected force values less than 40 N.

It should be noted that, according to several possible implementations related to the continuously detected force interval Fm, the algorithm can work on the entire spectrum. In an implementation option, in which the method is applied to a robotic system for micro-surgery, it is performed in the presence of the low forces present in such a context, i.e., forces less than 40 N, for example of the order of 10 N.

The advantage of such an implementation option is to ensure compensation during operation.

In accordance with an embodiment, the method is applied to a surgical instrument 20 which further comprises at least one transmission element 21, 22, 23, 24, 25, 26 operatively connected to a respective at least one actuation tendon 31, 32, 33, 34, 35, 36, and is operatively connectable to a respective motorized actuator 11, 12, 13, 14, 15, 16.

In such a case, therefore, the surgical instrument comprises a plurality of “transmission units”, each comprising an actuation tendon and a piston, in which preferably the tendon is fixed to the piston, and the respective motorized actuator acts by imparting a displacement on the piston of the transmission unit.

According to an implementation option, the surgical instrument comprises 6 transmission units, namely 6 tendons, 6 motorized actuators and 6 pistons.

According to an implementation option, each transmission unit (i.e., each motor-piston-tendon chain) is managed individually.

According to another implementation option, the antagonistic transmission units (and thus the antagonistic tendons) are managed in pairs.

In accordance with an implementation option, the step of imparting a movement and/or exerting a force comprises controlling the movement of each of the motorized actuators so that the movement of the transmission elements includes compensation due to the elongation or relaxation of the respective actuation tendons, based on both the estimated length variation of each of the actuation tendons and on the modulus and stiffness of said actuation tendons.

According to an implementation option, a reference kinematic zero condition is defined in the robotic system, by associating a virtual zero point with respect to which the movements imparted by the control means to the motorized actuators will be referred with respect to a stored reference position. In such a case, the step of imparting a movement and/or exerting a force on each of said transmission elements comprises calculating a corrected kinematic zero, which takes into account the compensation performed.

In accordance with an implementation option, the step of imparting a movement and/or exerting a force on each transmission element comprises applying a force to the transmission element by means of a double feedback-operated loop, wherein an elastic compensation correction is inserted in parallel to the displacement of the motorized actuator due to a movement kinematic mechanism.

In accordance with an embodiment, in the case in which the motorized actuators are stepper motor actuators, the position control is performed through a speed control, which, being known a working time unit, determines the position control.

In an implementation option of such an embodiment, the speed and position control is performed by means of a feedback-operated control loop, with a gain parameter (Kp) dimensioned so as to ensure the convergence of the compensation with a time constant lower than a maximum convergence time.

According to an implementation example, such maximum convergence time is less than one second, and preferably comprised in the interval between 100 ms and 200 ms.

According to an implementation option, the speed control comprises a kinematic component and a dynamic compensation component.

The dynamic compensation component receives the detected force Fm as an input, calculates the estimated displacement that is lost due to the elasticity of the actuation tendon, in accordance with the previously indicated formula, and, by means of a proportional controller tuned so as to have a dynamics conforming to the stability requirements, it generates a speed compensation contribution that is added to said speed kinematic component.

The sum of the aforesaid kinematic and dynamic speed contributions is provided as input to the motorized actuator to be controlled.

The controllers of the kinematic component and of the dynamic component are preferably in parallel.

In accordance with an embodiment, the position and/or speed control is performed in a common manner for a plurality of motorized actuators, for example by executing a joint control on each pair of antagonistic tendons, based on a common effective elasticity constant value, depending on conditions such as the position of the master or slave device, and/or the aging or state of the robotic system.

According to an embodiment, the position and/or speed control is performed only if the detected force Fm is lower than a maximum operating force value Fmax, and the execution of the method is inhibited when even only one of the motorized actuators detects a force greater than such a maximum operating force Fmax.

In fact, in such a case, a maximum force usable for safety limit is required to prevent the algorithm from diverging, i.e., in order to ensure convergence.

As explained above, alternatively, a controller can act on many tendons and take into account the state of the entire control system.

In an embodiment, the algorithm is deactivated when only one of the motors is above a certain threshold.

According to an embodiment, the elongation compensation parameters are determined in a controlled and variable manner depending on the pose of the articulated end effector 40 to take into account the different frictions related to the different poses.

For example, the winding angle of the tendons on the end effector links can be different between one tendon and the other antagonistic tendon, for example when near the stroke-end of a yaw degree of freedom. In such an embodiment, each tendon slides on convex curved surfaces of the links defining a contact path; the sum of all the contact paths of a tendon on convex curved surfaces of all the links (except the link to which it is fixed) at a certain time defines a winding angle.

Therefore, the link-tendon friction force between two antagonistic tendons of a pair is not always the same and varies based on the pose of the wrist. The algorithm recognizes the pose of the wrist and thus compensates for the elasticity on one tendon differently from the other.

In this embodiment, the algorithm is capable of linking the determination of a variable value K and/or the inhibition or operation of the compensation and/or the use of the compensation method on one tendon rather than another based on the known or calculated kinematic position of the wrist (end effector).

In an implementation option, experimental data is stored and/or the expected force on each tendon is mathematically computer-modeled depending on the pose of the wrist due to sliding frictions.

If an object is hit or moved (causing an increase in the external forces acting on the end effector), the detected force related to at least one tendon of a pair increases without activating the degree of freedom. In such a case, according to implementation examples, the method can change K and/or inhibit the operation of the compensation and/or use the compensation method on one tendon rather than another.

More generally, in an implementation example, it is determined whether an increase in force read by a sensor on a motorized actuator is due to movement of the wrist or due to external forces, and the compensation is adapted accordingly.

According to an embodiment, the method is applied to polymer actuation tendons preferably formed from intertwined polymer fibers.

According to an embodiment, the robotic system is a robotic system for micro-surgical teleoperation, and the surgical instrument is a micro-surgical instrument.

Again with reference to FIGS. 1-14, further details will be provided below, by way of non-limiting example, with reference to some particular embodiments of the method.

It should first be noted that, during teleoperation, the user is capable of controlling the instrument by virtue of the kinematic relationship which associates the displacement of a master device with the displacement of the motorized actuators (for example, six linear motorized actuators housed in the motor-box).

The controllability of the instrument is also ensured by the mechanical coupling between the aforesaid motorized actuators and the corresponding pistons present on the backend of the instrument itself.

A procedure is thus foreseen, called instrument “engagement”, which ensures the correct success of such a coupling. The engagement procedure is a necessary condition for each control operation of the instrument itself.

In this embodiment, the linear actuators present in the motor-box are capable of controlling three degrees of freedom (the aforementioned “Yaw”, “Pitch” and “Grip”) present in the wrist (i.e., end effector, or articulated end device) of the surgical instrument, through an appropriate transmission system consisting of a tendon system.

In particular, the controllable part of the microsurgical instrument consists of two tips, having a shared degree of freedom (Pitch) and a degree of freedom specific to each tip (Yaw). In this representation, the degree of freedom Grip can thus be defined as the difference between the commanded Yaw values of the two tips of the microsurgical instrument.

In such a mechanism, the coupling between instrument pistons and wrist is performed by means of two antagonistic tendons for each of the degrees of freedom previously described: two antagonistic tendons for the control of “Pitch” degree of freedom (shared by the two tips), and two antagonistic tendons for the control of “Yaw” degree of freedom of each of the two tips.

Approximately, the kinematic law which binds the six pistons of the instrument to the tips of the instrument considers the pistons of the motor-box connected to the two tips of the microsurgical instrument through non-stretchable tendons. As already observed in the previous description, the model accuracy necessary for a robust and efficient control of the instrument requires considering that the tendons are subject to reversible and irreversible deformations whenever stressed.

In particular, the actuation model that is here used provides that the instrument pistons are actuated by the six motors through a dedicated mechanical coupling which directly transforms the displacement of a motor into the displacement of the relative piston. Due to the not insignificant internal friction of the instrument, the displacement of a motor results in the application of a force on the relative piston. This command translates into cyclical elongation of the associated tendon.

While neglecting the dynamic components of motion and the presence of external forces, from the sole analysis of the system at static equilibrium, it is clear that a control system adapted to control the degrees of freedom of the instrument must take into account the following components added together:
displacement of the pistons such as to allow the wrist to reach the desired configuration;
the presence of a displacement of the aforesaid piston adapted to compensate for the elongations of the cable.

In this embodiment, by way of example, an algorithm is described which has the goal of ensuring kinematic congruence between master and slave devices, compensating for the elastic elongation of said tendons while respecting internal and external stability conditions.

In this context it is assumed that the plastic component is negligible or in any case compensated by other appropriately designed components of the control system.

The algorithm is based on the observability of the force applied by the actuator and the open loop and real-time compensation of the elastic loss calculated in accordance with Hooke's law. According to an implementation option, the algorithm acts during the robot teleoperation phase.

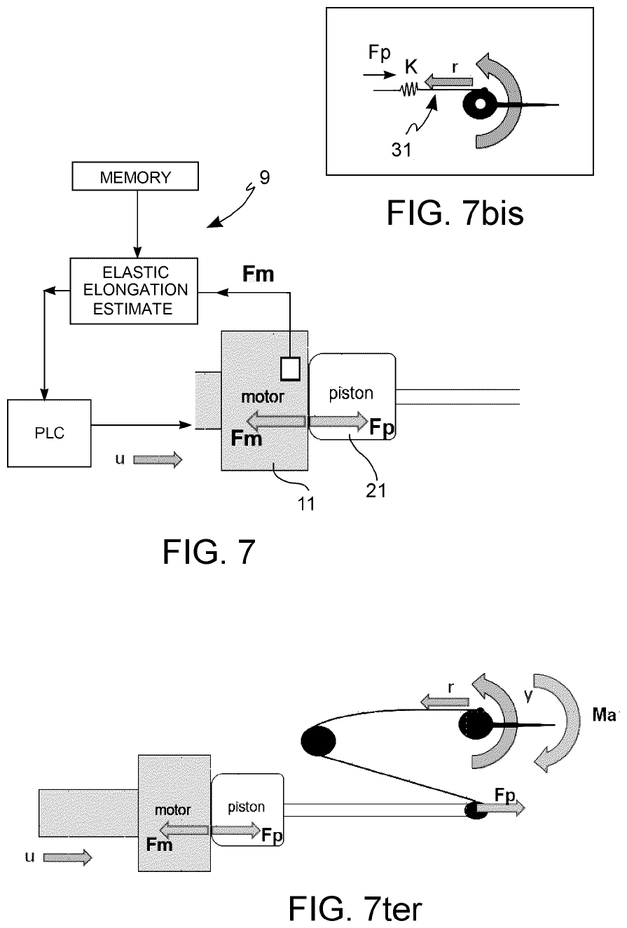

In accordance with the assumptions made in the previous paragraphs, each of the motor-piston-tendon systems can be considered as an isolated system and graphically modeled as in FIG. 7ter. As a first approximation, external forces on the tip or those caused by the antagonistic tendon are not considered.

In particular, the motor is appropriately dimensioned and controlled with a much faster dynamics than the model in question so that the dynamics of the motor can be modeled with a pure displacement in position. In this context, a controlled displacement u of the motor corresponds to a force Fp applied on the piston. The reaction force Fm=−Fp experienced by the motor during the movement is then acquired by an appropriate force sensor placed on the contact surface between motor and piston. It is assumed that the motor-piston coupling is always ensured.

The piston thus transfers the force Fp on the tendon which, working in traction, transfers the motion on the tip of the microsurgical instrument, where the force Fp (acting on the external surface of a non-zero radius rotoidal joint integral with the instrument tip) is balanced by a torque Ma summarizing the frictional forces present on the final rotoidal joint.

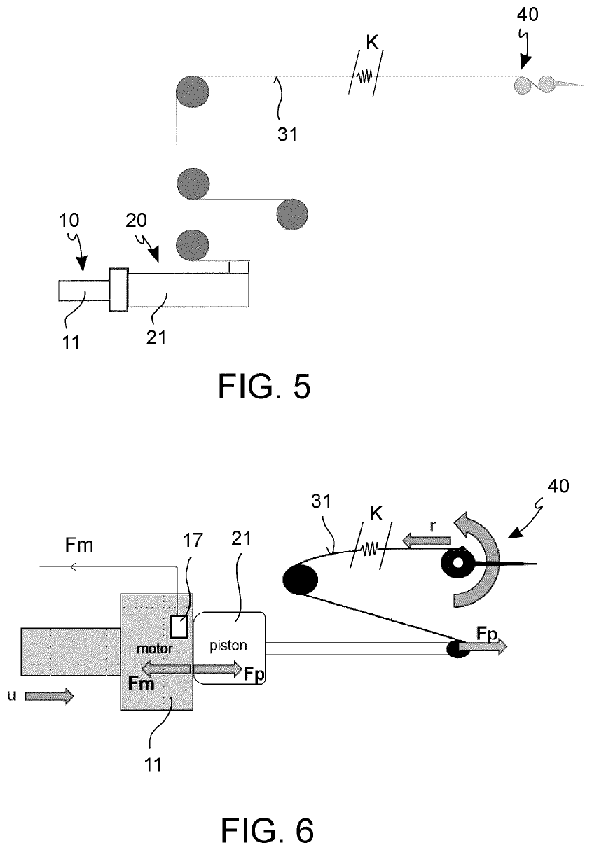

Referring to FIG. 5, since the friction introduced by the ball bearings (indicated as circles in the figure) is negligible with respect to Fp, then the force in the cable section between the bearings can be considered identical to that of the cable section towards the wrist.

Since the cable end section (comprised between the wrist pitch joint and the end node) is typically two orders of magnitude smaller than the total length of the cable, then the elongation of such an end section is negligible. Thus, the tendon elastic constant Kel and the constant force Fm along the entire cable can be considered, and the elongation is calculated as |Fm|/Kel:

$$r = u - \vert Fm \vert / Kel \qquad ( 1 )$$

where Kel is the tendon elastic constant, calculated in this case as Kel=E A L (where E=Young's modulus, A=cable section, L=cable length), and where r is the actual movement of the tip.

In an embodiment, Kel can be considered constant despite the polymer tendon being subject to cycling for the particular actuation method, and thus be governed by a variable Kel. This is due to the fact that, again for the particular type of actuation, each degree of freedom is controlled by two antagonistic tendons where it can be stated that only the tendon associated with the pushing motor contributes to the actual displacement in the desired direction while the antagonist is arranged at negligible forces in order to avoid a force component opposite the motion. It is thus possible to approximate the value Kel to a constant value which reflects the rising front of the hysteresis cycle characteristic of the polymer fiber in question.

|Fm| is the modulus of the force applied by the piston and measured by an appropriate force sensor, u is the position controlled by the motor and r is the displacement of a point on the external surface of the non-zero joint linked to the angular rotation of the tip y from the ratio y=r/R, with R being the radius of the rotoidal joint.

Starting from equation (1), we obtain:

$$y = ( u - \vert Fm \vert / Kel ) / R \qquad ( 2 )$$

In this context, the purpose of the control algorithm is to provide the motor with an appropriate control position u such as to:
1. minimize the error between the desired position x and the final position r; furthermore, it is necessary to ensure the asymptotic stability of the final position r, as well as the reachability thereof in a finite time;
2. ensure the internal stability of the system (finite u and Fm) in the borderline case in which, for arbitrarily long time intervals, there is no movement of the end effector at the desired target position variation, due for example to the presence of external forces or the prevalence of static friction.

Without losing in generality, it is assumed that the actuator is controlled by discrete speed control. The control dynamics of the actuator can be considered much faster than the dynamics of the system in question, therefore at any instant t (multiple of the discrete execution time of the algorithm) the position u can be considered equal to the integral in time of the speeds v sent to the motor up to that moment.

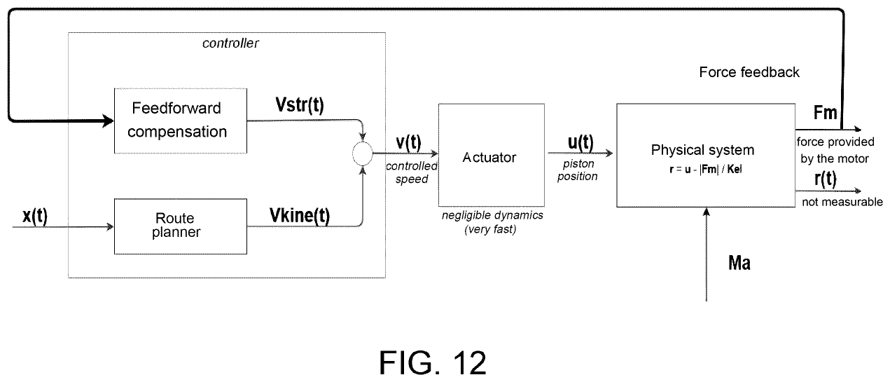

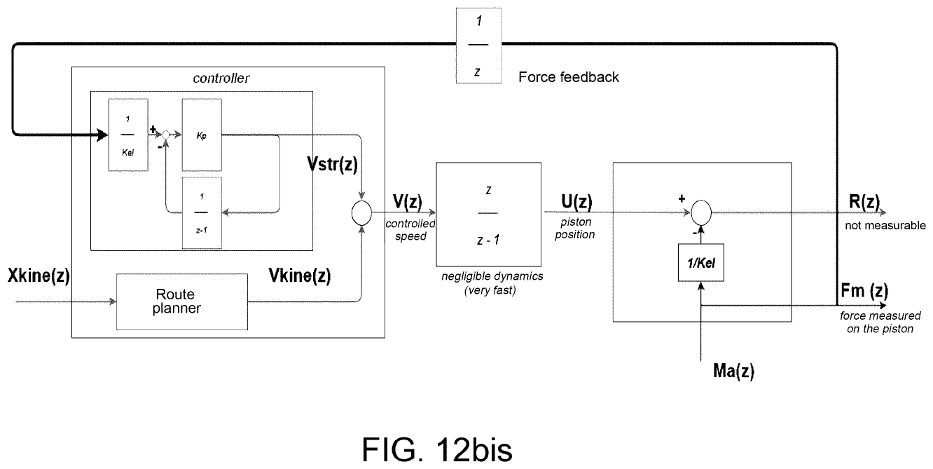

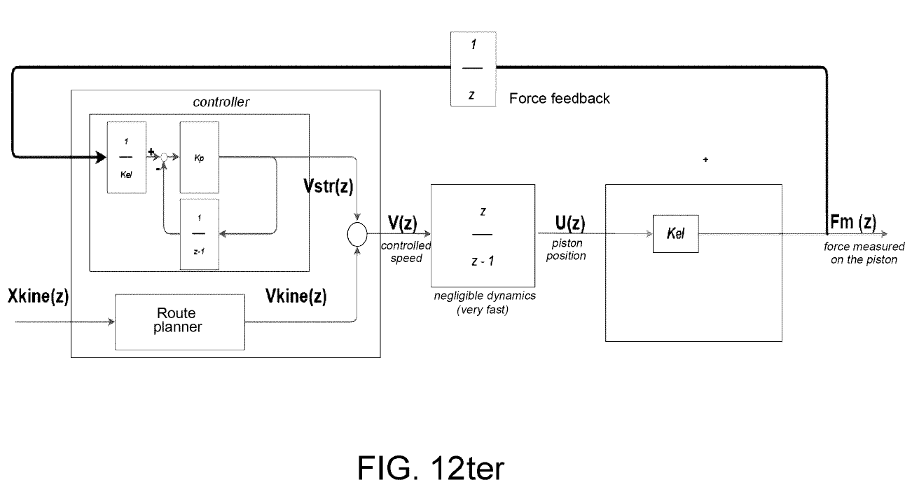

In FIG. 12 the control algorithm is presented in the case of Delta_r≠0 (system in motion). Similarly, in the case of Delta_r=0, the equation present in the Physical System block will be of the type Fm=Kel u.

The proposed algorithm, discretely modeled in the space of the Z transform, thus appears as shown in FIG. 12bis, in the case where Delta_r≠0, and as shown in FIG. 12ter, in the case where which Delta_r=0.

It should be noted that the algorithm, in the embodiment described herein, does not assume knowledge that the end effector is stationary or in motion, but proceeds according to the steps shown below. FIGS. 12, 12bis and 12ter must therefore be understood simply as an illustrative modeling described in the space of the Z transform.

In this embodiment, the proposed algorithm is intended to be executed on each of the motor-piston-transmission-tendon systems independently of each other.

Therefore, the algorithm consists of the following steps.
A) Real-time acquisition of the force modulus |Fm| exerted by the motor on the piston, for example by means of a load cell placed on the contact surface between motor and piston.
B) Assuming that the elongation experienced by the tendon is only of elastic type, it is calculated as: Δ_stretch=(|Fm|)/K, with tendon elastic constant K previously experimentally estimated (in the preferred form K=ø k_exp, where 0.7 >Ω>1.5, and thus with underestimation or overestimation of the elastic constant obtained experimentally).
C) The value Δ_stretch obtained is used as a reference for a proportional controller which at each control cycle returns the speed Vstr (z) component in feed-forward to be added to the kinematic trajectory commanded so as to compensate for the tendon elongation. It can be demonstrated that a properly calibrated proportional controller is a sufficient condition for obtaining stability of the system thus modeled.

Finally, in this embodiment, the management of the activation and deactivation of the algorithm is based on the state machine described in the following paragraphs.

The following parameters are involved.
K—Estimated tendon elastic constant (also defined above as “effective elastic constant value)

The elastic constant is a critical parameter because, in accordance with the proposed model, to ensure that the control variable u(z) is limited, the value much be greater than the real one (obtained experimentally) k_exp.

In particular, consider the case where the torque Me compensates for or exceeds the torque produced by the force Fm (no movement of the end effector, due for example to the presence of a strong static friction or the active presence of the antagonistic tendon, or an external force acting on the end effector). In this condition, the force Fm is dependent on the elongation of the cable, and thus

$$Fm = - Ku$$

where K=Ω k_exp. Thus, the force measured by the sensor is Fm=Ω k_exp u, in which Ω>1, from which the command to the motor u=Fm/(Ω k_exp).

In accordance with the model shown in FIG. 12ter, the controlled variable u(z) can be expressed as a function of the input xkine(z). The transfer function in the space of the Z transform of the system formed by Controller, Actuation and Physical System is as follows:

$$SYS 1 ( z ) = \frac{U ( z )}{X ( z )} = \frac{K [ z + ( {K}_{p} - 1 ) ]}{Kz + K ( {K}_{p} - 1 ) - {K}_{p} K}$$

having a single pole in

$$z = - {K}_{p} + 1 + \frac{k\_exp}{K} {K}_{p}$$

In accordance with the discrete systems analysis study, such a reference function is stable if the poles thereof are entirely contained in the unit circle, i.e., when the following relationship is valid:

$$\vert - {K}_{p} + 1 + \frac{k\_exp}{K} {K}_{p} \vert < 1$$

which in the case of K_p >0 is respected for values K >k_exp. As K increases, the stability margin of the system also increases. The physical meaning of having a K greater than the real k_exp is equivalent to compensating for less elongation than that present within the physical system.

The evaluation of the error introduced by the difference K−k_exp can be performed by analyzing the trend of the error if Delta_r≠0 (consider FIG. 12bis):

$$e = r - u$$

In this context, it is possible to express the error

$$E ( z ) = R ( z ) - Xkine ( z )$$

as MISO system function of the position Xkine(z) and the force Fm(z):

$$E ( z ) = {F}_{1} ( {X}_{kine} ( z ) ) + {F}_{2} ( {F}_{m} ( z ) ) - Xkine ( z ) \\ with \\ {F}_{1} = Xkine ( z ) \\ {F}_{2} = \frac{- K [ z + ( {K}_{p} - 1 ) ] + {K}_{p} k\_exp}{Kk\_exp [ z + ( {K}_{p} - 1 ) ]} {F}_{m} ( z ) \\ i . e . , \\ SYS 2 ( z ) = \frac{E ( z )}{{F}_{m} ( z )} = \frac{- K [ z + ( {K}_{p} - 1 ) ] + {K}_{p} k\_exp}{Kk\_exp [ z + ( {K}_{p} - 1 ) ]} {F}_{m} ( z )$$

For discrete variations of Fₘ(z), the extent of the error can be assessed using the final value theorem, i.e.,

$$( z - 1 ) SYS 2 ( z ) \frac{z}{z - 1} = \frac{k\_exp - K}{Kk\_exp}$$

which shows that complete error compensation is obtained only for K=k_exp. In this scenario, according to the proposed algorithm, the choice of K depends on the compromise between the amount of compensated tendon elongation and the robustness of the algorithm itself.

The fine adjustment of such a parameter thus depends on empirical considerations given by the variation of the real physical system with respect to the model used as well as the incidence of the forces at stake which oppose the movement of the tip of the microsurgical instrument.

It should be noted that the dynamics of the system reflect, as a first approximation, the dynamics of a system of the first order, i.e., an appropriate choice of control parameters will ensure the monotonous convergence of the position at the desired target.

Finally, it should be noted that, due to the nature of the controlled system, it is not possible to know the final force Fm to which the algorithm will converge a priori. Such a value Fm thus converges to a force value based on the dynamics of the trajectory applied, the physical plastic-elastic elongation features, the external disturbances of the system and the frictions inside the system.

Alternatively, or in addition, as mentioned above, the constant K can be chosen empirically regardless of the value of the actual tendon elastic constant. Accordingly, the value of the multiplicative parameter Q can be less than 1, and for example between 0.7 and 1. In an embodiment, the value of the multiplicative parameter Q belongs to the interval 0.7-1.5.

### K_p: Algorithm Convergence Speed.

With reference to the equations in the previous paragraph, it is evident that, if the static friction is exceeded, the transfer function SYS2(z) has a single pole present in position 1−K_p. In this context:

0<K_p<2 to make the control system stable.

The convergence speed increases by K_p→1. However, as the convergence speed increases, the phase margin of the system decreases.

If the static friction is not exceeded, the transfer function SYS1(z) has a single pole present in position

$$z = - {K}_{p} + 1 + \frac{k\_exp}{K} {K}_{p} .$$

In this context, for K_p→1 the dynamics is dominated by the ratio (k_exp)/K (where increasing values of K correspond to a higher convergence speed obtained at the expense of the precision of the algorithm itself). For small K_p, the dynamics of the algorithm is mainly governed by K_p.

From the considerations made in the previous paragraphs, it is therefore clear that, given the nature of the algorithm, it is not possible to compensate 100% of the elastic elongation if a given force is to be converged to in a finite time. The choice of the gain K_p and the accuracy of the estimate K meet both stability criteria and the guarantee of a convergence time which reflects the temporal usability requirements of the master-slave teleoperation.

An excessively slow algorithm would compromise the intuitiveness of the response of the microsurgical instrument to the commands provided by the operator. It should be noted that the controllable parameters v(z) and u(z) (instantaneous actuator speed and position, respectively) are subject to the physical amplitude and bandwidth constraints of the actuation system considered. Similarly, the observability of the variable F_m (force provided by an actuator at time t) is subject to the physical limitations of the chosen measuring instrument. Finally, the magnitude of the force F_m must be small enough not to affect the dynamics of the actuation system, as a first approximation commanded based on pure position. In this context, the choice of the parameters K and K_p must also take into account the constraints listed above.

In order to increase the robustness of the algorithm with non-modeled dynamics, the parameter Max Force is also introduced which defines the interval of the variable F_max within which the algorithm is capable of working.

Such non-modeled dynamics fall into the following families:
tendon friction along the entire transmission of the instrument and not fully applied at the end point;
application of a direct force on the tendon as a result of a torque applied to the tips of the instrument;
reversible and irreversible elongations of the tendon which do not comply with Hooke's law; the influence of such components can be mitigated by introducing additional compensation components in an open loop and, similarly to what has been done, studying the stability of the overall control system.

A possible configuration setup is given by way of example. Such a setup, in addition to being dependent on the parameters reported in the previous paragraphs, is uniquely associated with a type/class of microsurgical instrument. The identification of such a setup occurs experimentally, taking into account the criteria set out in the previous paragraphs:
Kp=0.02
K=25 N/mm
Max Force=14 N.

### Algorithm Activation Management

One of the peculiarities of the instrument in question is the ability to be able to grasp a surgical suture. The gripping concept is achieved by means of the simultaneous closure of the two tips belonging to the microsurgical instrument.

In this context, the gripping force is generated by controlling the instrument's degree of freedom of Grip, which is nothing more than the specular control of the degrees of freedom of Yaw of each of the two tips of the microsurgical system towards the objectives not reachable by the kinematics of the instrument itself (as they would require the mutual penetration of the tips themselves and thus the breakage of the kinematic constraints). Therefore, the system behaves as an open-loop force control in which the mechanical impedance of the two tendons is used to determine the gripping force as a function of the control variable u(z).

In this context, the need to compensate for cable elongations loses meaning, as such elongations will be the cause of the force present at the end effector.

Following the above considerations, it is important to ensure that the algorithm does not interfere with the quality of the grip in a pejorative manner. To this end, the steps of operability of the instrument in which the algorithm can be used will be analyzed below.

The steps (or states) of operability of the instrument can be summarized as follows.

HOLD: the state in which the instrument is engaged or in which there is kinematic continuity between the actuation system present in the motor-box and the pistons housed in the instrument. In such a state, the user does not have direct control over the instrument. The motors exert and maintain a force F_0 on the pistons. The kinematic position of the end effector is thus maintained at the expense of external forces acting on the tips.

OPERATION: the state in which the operator has direct control of the slave device through the use of a special master device, i.e., the operator is capable of moving the tip of the microsurgical instrument at will. In particular, as shown in FIG. 13, the user has the ability to modulate the gripping force by bringing the opening of the master device within the gripping interval of said “Operation Squeeze” region. Such an “Operation Squeeze” state can be divided into two sub-states called:
Operation Not Squeeze: the teleoperation is active and the operator keeps the master device above the gripping threshold;
Operation Squeeze: the teleoperation is active and the operator keeps the master device within the grip threshold.

The elastic compensation algorithm is active in the state called “Operation Not Squeeze”. In the “Operation Squeeze” state, the Feed-Forward component introduced by the algorithm is frozen, i.e., it is not possible to change the position offset provided by the algorithm until the return to the “Operation No Squeeze” step.

In an embodiment in which the two specific motors participating in the corresponding tip to close towards the inside of the instrument, the freezing of the offset is necessary since the algorithm, as described above, converges to an arbitrary force value and would thus hinder reaching the necessary force for gripping.

It is possible to exit the “Operation” state by means of the direct command of the operator who wants to interrupt the direct control of the slave device. Abandoning the “Operation” state implies the transition to the “Hold” state, crossing through the “Release” state (if in the “Operation Not Squeeze” sub-state) or “Freeze” state (if in the “Operation with Squeeze” sub-state).

RELEASE: intermediate state in the transition from Operation Not Squeeze to HOLD. In this state, the kinematic position component of the motors is preserved by instead removing the elastic compensation component from each motor, so that the teleoperation can be resumed in repeatable dynamic conditions.

FREEZE: intermediate state in the transition from Operation Squeeze to HOLD. In this state the motors are frozen in the current position so as to maintain the gripping force during the next HOLD step. In another implementation option, the FREEZE step involves the passage to a purely forceful control over the tendons which kinematically participate in the grip.

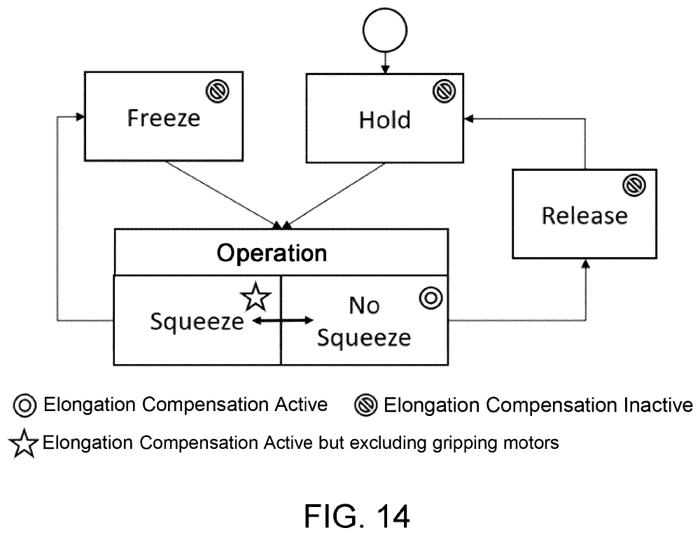

FIG. 14 shows a diagram depicting the previously described states and the passage from one state to another.

As can be seen in such a figure, the elastic compensation algorithm is only active during the teleoperation step (Operation). When the user controls the instrument using the master in the “Operation Not Squeeze” range, the algorithm is active on each motor, while if the user is in teleoperation using the master in the “Operation Squeeze” range, the position contribution of the elastic compensation of the two motors contributing to the closure of the tips is frozen at the entry into the state and the algorithm is deactivated on those motors. On the next entry into “Operation Not Squeeze”, the algorithm is reactivated on all the motors.

A robotic system for surgery, according to the present invention, is described below, again referring to FIGS. 1-14.

Such a robotic system for surgery comprises a surgical instrument 20, control means 9, at least one motorized actuator 11, 12, 13, 14, 15, 16, and force detection means.

The surgical instrument 20 comprises an articulated end effector 40 and at least one actuation tendon 31, 32, 33, 34, 35, 36, configured to actuate the articulated end effector 40.

The at least one motorized actuator 11, 12, 13, 14, 15, 16 is operatively connectable to a respective aforesaid at least one actuation tendon 31, 32, 33, 34, 35, 36 to impart an action, controlled by the control means 9, to the respective actuation tendon so as to determine a univocal correlation between at least one movement of one or more motorized actuators 11, 12, 13, 14, 15, 16 and a respective at least one movement of the articulated end effector 40.

The force detection means are configured to detect the force Fm exerted by at least one of the aforesaid one or more motorized actuators 11, 12, 13, 14, 15, 16, during an operating step of the surgical instrument.

The control means 9 are configured to carry out the following actions:
estimating, by means of a predefined mathematical model, based on the detected force Fm, a length variation of at least one of the aforesaid one or more actuation tendons 31, 32, 33, 34, 35, 36, due to elastic elongation of the actuation tendon;
using the estimated length variation for a position control of the one or more motorized actuators 11, 12, 13, 14, 15, 16, in which said position control comprises imparting a movement on said at least one of the aforesaid one or more motorized actuators 11, 12, 13, 14, 15, 16 taking into account the estimated length variation of at least one of the aforesaid one or more actuation tendons 31, 32, 33, 34, 35, 36, so as to reduce or cancel the error introduced by said elastic elongation between the position reached by the articulated end effector 40 and a desired nominal position of the articulated end effector 40.

According to an embodiment, the robotic system is a master-slave system in which the surgical instrument is a slave device controlled, according to a control mode, by a master device of the robotic system. The robotic system is configured to allow, in the absence of external forces, to minimize, in a finite time, the error between the pose commanded by the master device and the pose reached by the articulated end effector 40 of the slave device.

In accordance with an embodiment of the robotic system, the surgical instrument 20 comprises a plurality of actuation tendons 31, 32, 33, 34, 35, 36, and the robotic system for surgery comprises a respective plurality of motorized actuators 11, 12, 13, 14, 15, 16.

According to an implementation option of this embodiment, the aforesaid action of detecting a force is carried out on a plurality or on all the motorized actuators 11, 12, 13, 14, 15, 16, the aforesaid action of estimating is carried out with reference to a plurality or to all the actuation tendons 31, 32, 33, 34, 35, 36, and the aforesaid action of imparting is carried out on a plurality or on all the motorized actuators 11, 12, 13, 14, 15, 16.

According to an embodiment of the robotic system, the control means 9 are further configured to verify information related to the state of the robotic system; decide whether or not to perform the step of imparting a movement on a motorized actuator, to reduce and/or cancel and/or compensate for the error introduced by the elastic elongation, based on one or more conditions related to the state of the robotic system; and perform the aforesaid imparting action only if the aforesaid one or more conditions are met.

According to an implementation option, the master device is a hand-held, unconstrained master device adapted to be moved by an operator and manipulated by the operator according to a degree of freedom associated with the closing and/or gripping of the microsurgical slave instrument.

In such a case, when, during a teleoperation, the surgical instrument is in a gripping state, the aforesaid action of imparting a movement on a motorized actuator, to reduce and/or cancel and/or compensate for the error introduced by the elastic elongation, is inhibited or decreased according to a scaling factor between 0 and 1, for at least one of the motorized actuators connected to a respective at least one actuation tendon for the actuation of a gripping degree of freedom.

According to an embodiment of the system, said detection means of the force exerted by a motorized actuator 11, 12, 13, 14, 15, 16 comprise a respective force sensor or torque sensor operatively connected to the respective motorized actuator.

In accordance with an embodiment, the robotic system is configured such that the action of detecting a force Fm is performed continuously, with a detection frequency Fr and the aforesaid position control of the one or more motorized actuators is performed continuously, with a position control frequency Fcp.

The aforesaid detection frequency Fr and the aforesaid position control frequency Fcp are set so as to ensure a compensation of the elastic elongation in real time with a dynamics which cannot be perceived by the end user in real time, with a dynamics not perceivable by the user.

According to an implementation option, the aforesaid detection frequency Fr and position control frequency Fcp coincide, and are comprised in an interval between 100 Hz and 1000 Hz, and thus the compensation method is carried out at each period T, comprised in the interval between 1 and 10 ms, based on a force Fm detected at the same period.

According to an embodiment, the estimating action comprises estimating the length variation of an actuation tendon as the ratio between the modulus of the force detected Fm on the actuation tendon and an effective elastic constant value K, determined experimentally, or calculated or predetermined so as to ensure system response stability.

Further exemplary details on the formulas and parameters adopted for the above calculations have already been previously shown in the description of the method according to the invention.

According to an embodiment of the robotic system, the aforesaid surgical instrument 20 further comprises at least one transmission element 21, 22, 23, 24, 25, 26 operatively connected to a respective at least one actuation tendon 31, 32, 33, 34, 35, 36 and operatively connectable to a respective motorized actuator 11, 12, 13, 14, 15, 16.

In such a case, the action of imparting a movement and/or exerting a force comprises controlling the movement of each of the motorized actuators so that the movement of the transmission elements includes compensation due to the elongation or relaxation of the respective actuation tendons, based both on the estimated length variation of each of the actuation tendons and on the modulus and stiffness of the actuation tendons.

According to an implementation option, a reference kinematic zero condition is defined for the robotic system, associating a virtual zero point with respect to which the movements imparted by the control means to the motorized actuators will be referred with respect to a stored reference position. In such a case, the action of imparting a movement and/or exerting a force on each of the transmission elements comprises calculating a corrected kinematic zero, which takes into account the compensation performed.

According to an implementation option, the action of imparting a movement and/or exerting a force on each transmission element comprises applying a force to the transmission element by means of a double feedback-operated loop, wherein an elastic compensation correction is inserted in parallel to the displacement of the motorized actuator due to a movement kinematic mechanism.

In accordance with an embodiment of the robotic system, the motorized actuators are stepper motorized actuators, and the position control is performed through a speed control, which, a working time unit being known, determines the position control. In particular, the speed and position control is performed by means of a feedback-operated control loop, with a gain parameter Kp dimensioned so as to ensure the convergence of the compensation with a time constant lower than a maximum convergence time.

According to an implementation option, the speed control comprises a kinematic component and a dynamic compensation component.

The dynamic compensation component receives the detected force Fm as input, it calculates the estimated displacement lost due to the elasticity of the actuation tendon, in accordance with the formula reported in claim 16, and, by means of a proportional controller tuned so as to have a dynamics conforming to the stability requirements, it generates a speed compensation contribution which is added to said speed kinematic component.

The sum of the kinematic and dynamic speed contributions is supplied as input to the motorized actuator to be controlled. The controllers of the kinematic component and of the dynamic component are preferably in parallel.

According to an embodiment, the position and/or speed control is performed only if the detected force Fm is lower than a maximum operating force value Fmax, and the compensation is inhibited when even only one of the motorized actuators detects a force greater than said maximum operating force Fmax.

According to various possible embodiments, the robotic system is configured to carry out (in particular, under the control of the control means of the robotic system) a method according to any one of the previously described embodiments of the method.

As can be seen, the objects of the present invention as previously indicated are fully achieved by the method and system described above, by virtue of the features described above in detail, and as widely explained in the previous section “Summary of the invention”.

A person skilled in the art may make changes and adaptations to the embodiments of the method and system described above or can replace elements with others which are functionally equivalent to satisfy contingent needs without departing from the scope of protection of the appended claims. Each of the features described as belonging to a possible embodiment can be achieved irrespective of the other embodiments described.

LIST OF NUMERICAL REFERENCES

| | |
|---|---|
| 1 | Robotic system for teleoperated surgery |
| 2 | Slave assembly of the robotic system |
| 3 | Master console |
| 9 | Controller |
| 10 | Robotic system manipulator |
| 11, 12, 13, | Motorized manipulator actuators, or motors |
| 14, 15, 16 | |
| 17, 18 | Force sensors, or load cells |
| 19 | Sterile barrier |
| 20 | Surgical instrument |
| 21, 22, 23, | Surgical instrument transmission elements |
| 24, 25, 26 | |
| 27 | Shaft |
| 28 | Pocket |
| 29 | Surgical instrument backend, or surgical instrument |
| | transmission interface portion |
| 31, 32, 33, | Tendons |
| 34, 35, 36 | |
| 40 | End device, or articulated tip or end effector of the |
| | surgical instrument |
| 41, 42, | Links of the articulated end effector |
| 43, 44 | |
| x-x | Straight direction |
| r-r | Centerline |
| P, Y, G | Degree of freedom of the hinged tip, pitch, yaw, grip, |
| | respectively |
| k_exp | Experimentally obtained elastic constant, |
| K | Elastic constant used by the algorithm, |
| Ω | Ratio parameter between K and k_exp |
| Fm | Detected force |
| u | Controlled movement of the motor of the motorized actuator |

1. A method for controlling an articulated end effector actuated by one or more actuation tendons of a surgical instrument of a robotic system for surgery, executable during an operating phase of the surgical instrument,
wherein the surgical instrument comprises an articulated end effector and at least one actuation tendon, configured to actuate the articulated end effector,
and wherein the robotic system for surgery comprises, in addition to said surgical instrument, a controller and at least one motorized actuator, operatively connectable to a respective one of said at least one actuation tendon to impart an action to the respective actuation tendon, controlled by the controller, to determine a univocal correlation between at least one movement of the one or more motorized actuators and a respective at least one movement of the articulated end effector,
wherein the method comprises the steps of:
during said operating phase, detecting a force (Fm) exerted by at least one of said one or more motorized actuators;
estimating, by a predefined mathematical model, based on the detected force (Fm), a length variation of at least one of said one or more actuation tendons, due to elastic elongation of the actuation tendon;
using the estimated length variation for position control of the one or more motorized actuators, wherein said position control comprises:
imparting a movement to said at least one of said one or more motorized actuators taking into account the estimated length variation of said at least one of said one or more actuation tendons, to reduce or cancel error introduced by said elastic elongation between a position reached by the articulated end effector and a desired nominal position of the articulated end effector.

2. The method according to claim 1, wherein the robotic system is a master-slave system in which the surgical instrument is a slave device controlled, according to a control mode, by a master device of the robotic system,
wherein the method, in the absence of external forces, allows minimizing in a finite time an error between a pose commanded by the master device and a pose reached by the articulated end effector of the slave device,
and/or wherein the step of imparting takes into account a command action performed by a user.

3. The method according to claim 1, wherein the surgical instrument comprises a plurality of actuation tendons, and the robotic system for surgery comprises a respective plurality of motorized actuators,
wherein said step of detecting a force is carried out on a plurality or on all the motorized actuators, said step of estimating is carried out with reference to a plurality or to all the actuation tendons, said step of imparting is performed on a plurality or on all the motorized actuators,
wherein said actuation tendons are polymeric tendons comprising intertwined polymeric fibers,
wherein each of said one or more actuation tendons is operatively connected to both a respective motorized actuator of the robotic system for surgery and to said articulated end effector, to actuate a respective degree of freedom among one or more degrees of freedom of the articulated end effector, wherein at least one of said one or more actuation tendons actuates a rotational degree of freedom of the articulated end effector.

4. The method according to claim 1, comprising the further steps of:
verifying information related to the state of the robotic system;
deciding, by the controller, whether or not to perform said step of imparting a movement on a motorized actuator, to reduce and/or cancel and/or compensate for the error introduced by the elastic elongation, based on one or more conditions related to the state of the robotic system;
performing said step of imparting only if said one or more conditions are satisfied.

5. The method according to claim 4, wherein the master device is a hand-held, unconstrained master device adapted to be moved by an operator and manipulated by the operator according to a degree of freedom associated with closing and/or gripping of the microsurgical slave instrument,
wherein, when, during a teleoperation, the surgical instrument is in a gripping state, said step of imparting a movement on a motorized actuator, to reduce and/or cancel and/or compensate for the error introduced by the elastic elongation, is inhibited or decreased according to a scaling factor between 0 and 1, for at least one of the motorized actuators connected to a respective at least one actuation tendon for the actuation of a gripping degree of freedom.

6. The method according to claim 5, wherein said step of imparting a movement on a motorized actuator, to reduce and/or cancel and/or compensate for the error introduced by the elastic elongation, is inhibited or decreased according to a scaling factor between 0 and 1, for the two motorized actuators connected to respective two antagonistic actuation tendons of the grip closing degree of freedom, or for four motorized actuators connected to the four actuation tendons of the pairs of antagonistic actuation tendons of grip closing and grip opening degrees of freedom, or wherein said step of imparting a movement on a motorized actuator, to reduce and/or cancel and/or compensate for the error introduced by the elastic elongation, is inhibited or decreased according to a scaling factor between 0 and 1, for all the motorized actuators.

7. (canceled)

8. The method according to claim 4, wherein the master device is a hand-held, unconstrained master device adapted to be moved by an operator and manipulated by the operator according to a degree of freedom associated with closing and/or gripping of the microsurgical slave instrument,
wherein, at an end of a teleoperation, when the surgical instrument is in a gripping state and the gripping state is to be maintained, said step of imparting a movement on a motorized actuator, to reduce and/or cancel and/or compensate for the error introduced by the elastic elongation, is inhibited for all the motorized actuators connected to respective actuation tendons.

9. The method according to claim 1, wherein, when a teleoperation is exited in a non-gripping state, before re-entering a new teleoperation, the estimated length variation for each of the one or more actuation tendons, during a previous teleoperation, is reset.

10-12. (canceled)

13. The method according to claim 1, wherein the step of detecting a force is performed continuously, with a detection frequency, and said position control of the one or more motorized actuators is performed continuously, with a position control frequency,
wherein said detection frequency and said position control frequency are set to ensure a compensation of the elastic elongation in real time with a dynamics which cannot be perceived by a user in real time, with a dynamics which cannot be perceived by the user.

14. The method according to claim 2, wherein said detection frequency and position control frequency coincide, and are comprised in an interval between 100 Hz and 1000 Hz,
and wherein, the method is at each period T, comprised in an interval between 1 and 10 ms, based on a force detected at the same period.

15. The method according to claim 1, wherein the step of estimating comprises estimating the length variation of an actuation tendon as a ratio between a modulus of the detected force (Fm) on said actuation tendon and an effective elastic constant value (K), said effective elastic constant value (K) being determined experimentally, or calculated or pre-established to ensure system response stability.

16. The method according to claim 15, wherein the step of using the estimated length variation for a position control and the step of imparting a movement on the respective motorized actuator are carried out based on the formula:
$$u = \frac{Fm}{Ω Kel}$$
wherein said formula is specific for each motorized actuator to determine a specific control on each motorized actuator,
wherein u is the position that is controlled by the motorized actuator, Kel is the elastic constant of the actuation tendon, Ω is a multiplicative parameter, and/or
wherein the multiplicative parameter Ω is greater than 1, so that the effective elastic constant value K=Ω Kel used in the calculation is greater than the tendon elastic constant value by a factor equal to said multiplicative parameter Ω, and thus the effective tendon elastic constant value (K) is overestimated, and thus greater, than the elastic constant (Kel) of the same actuation tendon, and/or
wherein said multiplicative parameter is between 0.7 and 1.5, and/or
wherein said effective elastic constant value K, and therefore also the multiplicative parameter Ω, are determined in a variable manner, depending on a state of the robotic system, and/or on spatial conditions of the master device and/or a slave device and/or on teleoperation permanence time.

17-20. (canceled)

21. The method according to claim 1, wherein said surgical instrument further comprises at least one transmission element operatively connected to a respective at least one actuation tendon and operatively connectable to a respective motorized actuator, and
wherein the step of imparting a movement and/or exerting a force comprises controlling the movement of each of the motorized actuators so that the movement of the transmission elements includes compensation due to elongation or relaxation of the respective actuation tendons, based on both an estimated length variation of each of the actuation tendons and a modulus and stiffness of said actuation tendons.

22. (canceled)

23. The method according to claim 13, wherein a reference kinematic zero condition is defined in the robotic system, associating a virtual zero point with respect to which the movements imparted by the controller to the motorized actuators will be referred with respect to a stored reference position,
and wherein the step of imparting a movement and/or exerting a force on each of said transmission elements comprises calculating a corrected kinematic zero, which takes into account the compensation performed.

24. The method according to claim 13, wherein the step of imparting a movement and/or exerting a force on each transmission element comprises applying a force to the transmission element by a double feedback-operated loop, wherein an elastic compensation correction is inserted in parallel to displacement of the motorized actuator due to a movement kinematic mechanism.

25. The method according to claim 1, wherein the motorized actuators are stepper motorized actuators, and wherein the position control is performed through a speed control, which, being known a working time unit, determines the position control,
wherein the speed and position control is performed by a feedback-operated control loop, with a gain parameter dimensioned to ensure convergence of the compensation with a time constant lower than a maximum convergence time.

26-27. (canceled)

28. The method according to claim 15, wherein the speed control comprises a kinematic component and a dynamic compensation component,
wherein the dynamic compensation component receives the detected force (Fm) as input, and calculates estimated displacement lost due to elasticity of the actuation tendon, in accordance with the formula:
$$u = \frac{Fm}{Ω Kel}$$
wherein u is the position that is controlled by the motorized actuator, Kel is the elastic constant of the actuation tendon, is a multiplicative parameter,
and, by a proportional controller tuned to have dynamics conforming to the stability requirements, the dynamic compensation component generates a speed compensation contribution which is added to said speed kinematic component,
wherein a sum of said kinematic and dynamic speed contributions is supplied as input to the motorized actuator to be controlled,
and wherein the controllers of the kinematic component and of the dynamic component are in parallel.

29. The method according to claim 1, wherein the position and/or speed control is performed in a common manner for a plurality of motorized actuators, by performing a joint control on each pair of antagonistic tendons, based on a common effective elasticity constant value, depending on conditions comprising a position of the master or slave device, and/or aging or state of the robotic system, and/or
wherein the position and/or speed control is performed only if the detected force is lower than a maximum operating force value, and wherein the method is inhibited when even only one of the motorized actuators detects a force greater than said maximum operating force.

30. (canceled)

31. The method according to claim 1, wherein elongation compensation parameters are determined in a controlled and variable manner depending on a pose of the articulated end effector to take into account different frictions related to different poses.

32. (canceled)

33. A robotic system for surgery, comprising:
a surgical instrument comprising an articulated end effector and at least one actuation tendon, configured to actuate the articulated end effector;
a controller;
at least one motorized actuator, operatively connectable to a respective said at least one actuation tendon to impart an action to the respective actuation tendon, controlled by the controller, so as to determine a univocal correlation between at least one movement of one or more motorized actuators and a respective at least one movement of the articulated end effector;
a force detector configured to detect a force exerted by at least one of said one or more motorized actuators, during an operating phase of the surgical instrument;
wherein the controller is configured to carry out the following actions:
estimating, by a predefined mathematical model, based on the detected force, a length variation of at least one of said one or more actuation tendons, due to elastic elongation of the actuation tendon;
using the estimated length variation for a position control of the one or more motorized actuators, wherein said position control comprises imparting a movement to said at least one of said one or more motorized actuators taking into account the estimated length variation of said at least one of said one or more actuation tendons to reduce or cancel an error introduced by said elastic elongation between a position reached by the articulated end effector and a desired nominal position of the articulated end effector.

34-51. (canceled)
